(19) United States
(12) Patent Application Publication
(10) Pub. No.: US 2015/0265592 A1
(43) Pub. Date: Sep. 24, 2015

(54) USE OF HIGH DOSE LAQUINIMOD FOR TREATING MULTIPLE SCLEROSIS

(71) Applicant: Dan Bar-Zohar, Ramat-Gan (IL)

(72) Inventor: Dan Bar-Zohar, Ramat-Gan (IL)

(73) Assignee: TEVA PHARMACEUTICAL INDUSTRIES, LTD., Petach-Tikva (IL)

(21) Appl. No.: 14/731,971

(22) Filed: Jun. 5, 2015

Related U.S. Application Data
(63) Continuation of application No. 13/874,537, filed on May 1, 2013.
(60) Provisional application No. 61/641,389, filed on May 2, 2012.

(51) Int. Cl. A61K 31/4704; A61K 45/06

(52) U.S. Cl. CPC A61K 31/4704; A61K 45/06

(57) ABSTRACT

Disclosed herein are methods of treating a human patient afflicted with multiple sclerosis or presenting a clinically isolated syndrome, methods for treating a human subject by providing neuroprotection to the human subject, and methods of treating a human patient afflicted with multiple sclerosis or presenting a clinically isolated syndrome by increasing the time to confirmed disease progression, increasing the time to confirmed relapse or reducing brain atrophy in the human patient, comprising orally administering to the human patient or subject a daily dose of about 1.2 mg laquinimod or a pharmaceutically acceptable salt thereof. The subject invention also provides a pharmaceutical oral unit dosage form of about 1.2 mg laquinimod or a pharmaceutically acceptable salt thereof and a pharmaceutically acceptable carrier for use in treating a human patient afflicted with multiple sclerosis or presenting a clinically isolated syndrome, for use in treating a human subject by providing neuroprotection to the human subject, or for use treating a human patient afflicted with multiple sclerosis or presenting a clinically isolated syndrome by increasing the time to confirmed disease progression, increasing the time to confirmed relapse or reducing brain atrophy in the human patient.

This application claims benefit of U.S. Provisional Application No. 61/641,389, filed May 2, 2012, the entire content of which is hereby incorporated by reference herein.

Throughout this application, various publications are referred to by first author and year of publication. Full citations for these publications are presented in a References section immediately before the claims. Disclosures of the publications cited in the References section in their entireties are hereby incorporated by reference into this application in order to more fully describe the state of the art as of the date of the invention described herein.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care. (EMEA Guideline, 2006)

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS. (Bjartmar, 2002) In addition to the inflammatory phase in MS, axonal loss occurs early in the course of the disease and can be extensive over time, leading to the subsequent development of progressive, permanent, neurologic impairment and, frequently, severe disability. (Neuhaus, 2003) Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction. (EMEA Guideline, 2006)

Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination. (Neuhaus, 2003; Noseworthy, 2000)

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria. (McDonald, 2001) The McDonald Criteria was revised in March 2005 (Polman, 2005) and again in 2010 (Polman, 2011) by an international panel.

Intervention with disease-modifying therapy at relapsing stages of MS is suggested to reduce and/or prevent accumulating neurodegeneration. (Hohlfeld, 2000; De Stefano, 1999) There are currently six disease-modifying treatments for MS approved by regulatory agencies of various countries: Fingolimod (Gilenya®), Interferon beta-1a (Avonex®, CinnoVex®, ReciGen® and Rebif®), interferon beta-1b (Betaseron® and Betaferon®), glatiramer acetate (Copaxone®), mitoxantrone (Novantrone®) and natalizumab (Tysabri®). The interferons and glatiramer acetate are delivered by frequent injections, varying from once-per-day for glatiramer acetate to once-per-week (but intra-muscular) for Avonex®. Natalizumab and mitoxantrone are given by IV infusion at monthly intervals. Most of them are believed to act as immunomodulators. Mitoxantrone and natalizumab are believed to act as immunosuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled. (EMEA Guideline, 2006)

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Laquinimod

Laquinimod sodium is a novel synthetic compound with high oral bioavailability, which has been suggested as an oral formulation for the treatment of MS. (Polman, 2005; Sandberg-Wollheim, 2005)

Studies have shown laquinimod to reduce development of active MRI lesions in relapsing MS. (Polman, 2005) However, the clinical significance of MRI brain lesion reduction alone is still unsettled. Although MRI lesions are used as the primary outcome measure in some studies, others have suggested that correlation between MRI abnormalities and clinical disease activity in patients with RRMS is weak and that such measurement should be used as secondary outcomes rather than as surrogate markers of clinical responses. (Rudick, 1999; Miki, 1999; Barkhof, 1999) Further, according to pharmaceutical regulatory bodies such as the European Medicines Agency (EMEA), the correlation between MRI results and clinical outcomes has not been proved strong enough so as to accept MRI results as validated surrogate endpoint in pivotal studies. Therefore, according to the EMEA, the relevant efficacy parameter for clinical trials is the accumulation of disability and relapse rate (for RRMS). (EMEA Guideline, 2006) Thus, relapse rate and progression of disability are the currently accepted indicators of the effectiveness of a treatment for RRMS, but these have not previously been established for laquinimod.

The EMEA MS clinical trials guideline further states that the annual relapse rate in RRMS is usually low and that, generally, progression of disability takes years. Consequently, confirmatory studies with products intended to modify the course of the disease should be large scale and long enough to have a substantial proportion of patients suffering relapses or showing progression of disability. Two years is considered the minimum duration to demonstrate efficacy. (EMEA Guideline, 2006)

Furthermore, existing literature reached different conclusions as to the effective dose of laquinimod for the treatment of MS. The 0.3 mg/day oral dose was shown to reduce development of active MRI lesions in relapsing MS (which includes RRMS and SPMS) in one study (Polman, 2005), while another study showed the same dose to have neither MRI nor clinical effect as compared to placebo. (Comi, 2007)

### SUMMARY OF THE INVENTION

The subject invention provides a method of treating a human patient afflicted with multiple sclerosis or presenting a clinically isolated syndrome, the method comprising orally administering to the human patient laquinimod or a pharmaceutically acceptable salt thereof at a daily dose of about 1.2 mg laquinimod so as to thereby treat the human patient.

The subject invention also provides a method for treating a human subject by providing neuroprotection to the human subject comprising orally administering to the human subject a daily dose of about 1.2 mg laquinimod or a pharmaceutically acceptable salt thereof so as to thereby treat the human subject by providing neuroprotection to the human subject.

The subject invention also provides a method of treating a human patient afflicted with multiple sclerosis or presenting a clinically isolated syndrome by increasing the time to confirmed disease progression, increasing the time to confirmed relapse or reducing brain atrophy in the human patient, the method comprising orally administering to the patient laquinimod or a pharmaceutically acceptable salt thereof at a daily dose of about 1.2 mg laquinimod so as to thereby treat the human patient by increasing the time to confirmed disease progression, increasing the time to confirmed relapse or reducing brain atrophy in the human patient.

The subject invention also provides a pharmaceutical oral unit dosage form of about 1.2 mg laquinimod or a pharmaceutically acceptable salt thereof and a pharmaceutically acceptable carrier for use in treating a human patient afflicted with multiple sclerosis or presenting a clinically isolated syndrome, a pharmaceutical oral unit dosage form of about 1.2 mg laquinimod or a pharmaceutically acceptable salt thereof and a pharmaceutically acceptable carrier for use in treating a human subject by providing neuroprotection to the human subject, and a pharmaceutical oral unit dosage form of about 1.2 mg laquinimod or a pharmaceutically acceptable salt thereof and a pharmaceutically acceptable carrier for use treating a human patient afflicted with multiple sclerosis or presenting a clinically isolated syndrome by increasing the time to confirmed disease progression, increasing the time to confirmed relapse or reducing brain atrophy in the human patient.

### DETAILED DESCRIPTION OF THE INVENTION

The subject invention provides a method of treating a human patient afflicted with multiple sclerosis or presenting a clinically isolated syndrome, the method comprising orally administering to the human patient laquinimod or a pharmaceutically acceptable salt thereof at a daily dose of about 1.2 mg laquinimod so as to thereby treat the human patient.

In one embodiment, the administration laquinimod is effective to alleviate a symptom of or a condition associated with multiple sclerosis. In another embodiment, the administration of laquinimod is effective to increase the time to confirmed disease progression, increase the time to confirmed relapse, reduce brain atrophy, reduce relapse rate, reduce rate of confirmed relapses requiring hospitalization and/or IV steroids, reduce the accumulation of disability, reduce or inhibit progression of the level of fatigue, improve or inhibit deterioration of the functional status, improve or inhibit deterioration of the general health, reduce MRI-monitored disease activity or reduce cognitive impairment in the human patient.

In one embodiment, the administration of laquinimod is effective to increase the time to confirmed disease progression in the human patient. In another embodiment, confirmed disease progression is measured by Kurtzke Expanded Disability Status Scale (EDSS) score.

In one embodiment, the patient had an EDSS score of 0-5.5 prior to administration of laquinimod. In another embodiment, the patient had an EDSS score of 5 or less prior to administration of laquinimod. In another embodiment, confirmed disease progression is at least a 1 point increase of the EDSS score. In one embodiment, the patient had an EDSS score of 5.5 or greater prior to administration of laquinimod. In another embodiment, confirmed disease progression is at least a 0.5 point increase of the EDSS score.

In one embodiment, the time to confirmed disease progression is increased by 20-60%. In another embodiment, the time to confirmed disease progression is increased by 30-50%. In another embodiment, the time to confirmed disease progression is increased by at least 30%. In another embodiment, the time to confirmed disease progression is increased by at least 40%. In yet another embodiment, the time to confirmed disease progression is increased by at least 50%.

In one embodiment, the administration of laquinimod is effective to increase time to confirmed relapse in the human patient. In another embodiment, the time to confirmed relapse is increased by at least 20%. In another embodiment, the time to confirmed relapse is increased by at least 30%. In another embodiment, the time to confirmed relapse is increased by at least 40%. In another embodiment, the time to confirmed relapse is increased by at least 50%.

In one embodiment, the administration of laquinimod is effective to reduce brain atrophy in the human patient. In another embodiment, brain atrophy is reduced by 15-40%. In another embodiment, brain atrophy is reduced by at least 20%. In another embodiment, brain atrophy is reduced by at least 30%. In another embodiment, brain atrophy is reduced by at least 40%. In yet another embodiment, brain atrophy is reduced by at least 50%.

In one embodiment, the administration of laquinimod is effective to reduce relapse rate in the human patient. In another embodiment, the relapse rate is reduced by at least 20%. In another embodiment, the relapse rate is reduced by at least 30%. In another embodiment, the relapse rate is reduced by at least 40%. In another embodiment, the relapse rate is reduced by at least 50%. In another embodiment, the relapse rate is reduced by at least 60%. In yet another embodiment, the relapse rate is reduced by at least 70%.

In one embodiment, the administration of laquinimod is effective to reduce the accumulation of disability in the human patient. In another embodiment, the accumulation of disability is assessed by the timed 25-foot walk (T25FW). In another embodiment, the accumulation of disability is assessed by the progression of the subject's MS Functional Composite (MSFC) score. In another embodiment, patient's MSFC score improves within 3 months of first laquinimod treatment. In another embodiment, patient's MSFC score improves within 6 months of first laquinimod treatment. In another embodiment, patient's MSFC score improves within 12 months of first laquinimod treatment. In another embodiment, patient's MSFC score improves within 18 months of first laquinimod treatment. In another embodiment, patient's MSFC score improves within 24 months of first laquinimod treatment.

In one embodiment, the administration of laquinimod reduces patient's risk for a confirmed disease progression by at least 30%, compared to a patient not receiving the laquinimod treatment. In another embodiment, the administration of laquinimod reduces patient's risk for a confirmed disease progression by at least 35%, compared to a patient not receiving the laquinimod treatment. In another embodiment, the administration of laquinimod reduces patient's risk for a confirmed disease progression by at least 40%, compared to a patient not receiving the laquinimod treatment. In an embodiment, the risk reduction occurred within 3 months of first laquinimod treatment. In another embodiment, the risk reduction occurred within 6 months of first laquinimod treatment. In another embodiment, the risk reduction occurred within 12 months of first laquinimod treatment. In another embodiment, the risk reduction occurred within 18 months of first laquinimod treatment. In another embodiment, the risk reduction occurred within 24 months of first laquinimod treatment.

In one embodiment, the administration of laquinimod is effective to reduce or inhibit progression of the level of fatigue in the human patient. In an embodiment, the level of fatigue is assessed by the patient's Modified Fatigue Impact Scale (MFIS) score. In another embodiment, the administration of laquinimod decreased the human patient's MFIS score, compared to a patient not receiving the laquinimod treatment. In another embodiment, the administration of laquinimod decreased the human patient's MFIS score, compared to the patient at the start of the laquinimod treatment. In yet another embodiment, the MFIS score decreased within 24 months of the start of laquinimod treatment

In one embodiment, the administration of laquinimod is effective to improve or inhibit deterioration of the functional status in the human patient. In another embodiment, the functional status of the patient is measured by the patient's Short-Form General Health survey (SF-36) Subject-Reported Questionnaire score. In another embodiment, the administration of laquinimod decreased the human patient's SF-36 score, compared to a patient not receiving the laquinimod treatment. In another embodiment, the administration of laquinimod decreased the human patient's SF-36 score, compared to the patient at the start of the laquinimod treatment. In another embodiment, the patient's SF-36 mental component summary score (MSC) is decreased. In another embodiment, the patient's SF-36 physical component summary score (PSC) is decreased. In yet another embodiment, the SF-36 score is decreased within 24 months of the start of laquinimod treatment.

In one embodiment, the administration of laquinimod is effective to improve or inhibit deterioration of the general health in the human patient. In another embodiment, the general health of the patient is assessed by the patient's EQ-5D Standardized Questionnaire score. In another embodiment, the administration of laquinimod increased the human patient's EQ-5D score, compared to a patient not receiving the laquinimod treatment. In another embodiment, the administration of laquinimod increased the human patient's EQ-5D score, compared to the patient at the start of the laquinimod treatment. In another embodiment, the EQ-5D score increased within 24 months of the start of laquinimod treatment.

In one embodiment, the administration of laquinimod is effective to reduce MRI-monitored disease activity in the human patient.

In an embodiment, the MRI-monitored disease activity is assessed by the number of GdE-T1 lesions, the number of new T2 lesions, the number of new T1 hypointense lesions (black holes), change in T2 lesions volume, change in GdE-T1 lesions volume or change in T1 hypointense lesions volume (black holes). In another embodiment, the MRI-monitored disease activity is the cumulative number of enhancing lesions on T₁-weighted images, the cumulative number of new hypointense lesions on T₁-scans, and the cumulative number of new T₂ lesions. In another embodiment, the MRI-monitored disease activity is the mean cumulative number of Gd-Enhancing lesions, Gd-enhanced lesion counts, change in T₂ visible lesion or change in brain volume.

In one embodiment, the administration of laquinimod is effective to reduce cognitive impairment in the human patient. In another embodiment, the cognitive impairment is assessed by the Symbol Digit Modalities Test (SDMT) score.

In one embodiment, the patient had disease duration of at least 6 months prior to starting laquinimod treatment.

In one embodiment, the laquinimod is administered as monotherapy for multiple sclerosis. In another embodiment, the laquinimod is administered as adjunct therapy with an other multiple sclerosis treatment. In another embodiment, the other relapsing-remitting multiple sclerosis treatment is administration of interferon beta 1-a, interferon beta 1-b, glatiramer acetate, mitoxantrone, natalizumab, dialkyl fumarate or fingolimod. In yet another embodiment, the human patient is afflicted with relapsing-remitting multiple sclerosis.

The subject invention also provides a method for treating a human subject by providing neuroprotection to the human subject comprising orally administering to the human subject a daily dose of about 1.2 mg laquinimod or a pharmaceutically acceptable salt thereof so as to thereby treat the human subject by providing neuroprotection to the human subject.

In an embodiment, the administration of laquinimod reduces neuronal dysfunction, reduces neuronal injury, reduces neuronal degeneration, and/or reduces neuronal apoptosis. In another embodiment, the administration of laquinimod reduces neuronal dysfunction in the Central Nervous System, reduces neuronal injury in the Central Nervous System, reduces neuronal degeneration in the Central Nervous System, an/or reduces neuronal apoptosis in the Central Nervous System. In yet another embodiment, the administration of laquinimod reduces neuronal dysfunction in the peripheral nervous system (PNS) consists, reduces neuronal injury in the peripheral nervous system (PNS), reduces neuronal degeneration in the peripheral nervous system (PNS), an/or reduces neuronal apoptosis in the peripheral nervous system (PNS).

In one embodiment, the method of any of the above comprises orally administering to the patient laquinimod or a pharmaceutically acceptable salt thereof at a daily dose of substantially 1.2 mg laquinimod. In another embodiment, the method comprises orally administering to the patient laquinimod or a pharmaceutically acceptable salt thereof at a daily dose of 1.2 mg laquinimod. In another embodiment, the laquinimod is administered in the form of laquinimod sodium.

In one embodiment, the administration is for a period of greater than 24 weeks. In another embodiment of any of the methods described herein, the administration is for a period of greater than 36 weeks. In another embodiment of any of the methods described herein, the administration is for a period of greater than 48 weeks.

The subject invention also provides a method of treating a human patient afflicted with multiple sclerosis or presenting a clinically isolated syndrome by increasing the time to confirmed disease progression, increasing the time to confirmed relapse or reducing brain atrophy in the human patient, the method comprising orally administering to the patient laquinimod or a pharmaceutically acceptable salt thereof at a daily dose of about 1.2 mg laquinimod so as to thereby treat the human patient by increasing the time to confirmed disease progression, increasing the time to confirmed relapse or reducing brain atrophy in the human patient.

In one embodiment, the administration laquinimod is effective to increase the time to confirmed disease progression in the human patient. In another embodiment, the administration of laquinimod is effective to increase the time to confirmed relapse in the human patient. In yet another embodiment, the administration of laquinimod is effective to reduce brain atrophy in the human patient.

In one embodiment, the laquinimod is administered as monotherapy for multiple sclerosis. In another embodiment, the laquinimod is administered as adjunct therapy with an other multiple sclerosis treatment. In yet another embodiment, the other relapsing-remitting multiple sclerosis treatment is administration of interferon beta 1-a, interferon beta 1-b, glatiramer acetate, mitoxantrone, natalizumab, dialkyl fumarate or fingolimod.

In one embodiment, the human patient is afflicted with relapsing-remitting multiple sclerosis. In another embodiment, the method comprises orally administering to the patient laquinimod or a pharmaceutically acceptable salt thereof at a daily dose of substantially 1.2 mg laquinimod. In another embodiment, the method comprises orally administering to the patient laquinimod or a pharmaceutically acceptable salt thereof at a daily dose of 1.2 mg laquinimod. In yet another embodiment, the laquinimod is administered in the form of laquinimod sodium.

In one embodiment, the administration is for a period of greater than 24 weeks. In another embodiment of any of the methods described herein, the administration is for a period of greater than 36 weeks. In another embodiment of any of the methods described herein, the administration is for a period of greater than 48 weeks.

In an embodiment, the laquinimod or pharmaceutically acceptable salt thereof is administered in the form of a tablet. In another embodiment, the laquinimod or pharmaceutically acceptable salt thereof is administered in the form of a capsule.

The subject invention also provides a pharmaceutical oral unit dosage form of about 1.2 mg laquinimod or a pharmaceutically acceptable salt thereof and a pharmaceutically acceptable carrier for use in treating a human patient afflicted with multiple sclerosis or presenting a clinically isolated syndrome.

The subject invention also provides a pharmaceutical oral unit dosage form of about 1.2 mg laquinimod or a pharmaceutically acceptable salt thereof and a pharmaceutically acceptable carrier for use in treating a human subject by providing neuroprotection to the human subject.

The subject invention also provides a pharmaceutical oral unit dosage form of about 1.2 mg laquinimod or a pharmaceutically acceptable salt thereof and a pharmaceutically acceptable carrier for use treating a human patient afflicted with multiple sclerosis or presenting a clinically isolated syndrome by increasing the time to confirmed disease progression, increasing the time to confirmed relapse or reducing brain atrophy in the human patient.

In one embodiment, the pharmaceutical oral unit dosage form contains substantially 1.2 mg laquinimod. In another embodiment, the pharmaceutical oral unit dosage form contains 1.2 mg laquinimod.

In an embodiment, the pharmaceutical oral unit dosage form is in the form of a tablet. In another embodiment, the pharmaceutical oral unit dosage form is in the form of a capsule.

The subject invention also provides a method of reducing the likelihood that a relapsing-remitting multiple sclerosis human patient would experience a confirmed relapse within a predetermined time period, the method comprising orally administering to the patient laquinimod or a pharmaceutically acceptable salt thereof at a daily dose of about 1.2 mg laquinimod so as to thereby reduce the likelihood that the relapsing-remitting multiple sclerosis human patient would experience a confirmed relapse within the predetermined period. In one embodiment, the predetermined time period is 12 months. In another embodiment, the predetermined time period is 24 months.

In one embodiment, the relapse rate or the likelihood (risk) of relapse is reduced by at least 20%, compared to a patient not receiving the laquinimod treatment. In another embodiment, the relapse rate or the likelihood (risk) of relapse is reduced by at least 25%, compared to a patient not receiving the laquinimod treatment. In another embodiment, the relapse rate or the likelihood (risk) of relapse is reduced by at least 30%, compared to a patient not receiving the laquinimod treatment. In yet another embodiment, the relapse rate or the likelihood (risk) of relapse is reduced by at least 70%, compared to a patient not receiving the laquinimod treatment.

In one embodiment, the relapse is a severe relapse requiring hospitalization or IV-steroid treatment. In another embodiment, the patient's annualized rate of relapses requiring hospitalization is reduced by at least 20%, or at least 25%, compared to a patient not receiving the laquinimod treatment.

The subject invention further provides a method of decreasing the severity or duration of a relapse in a relapsing-remitting multiple sclerosis human patient, the method comprising orally administering to the patient laquinimod or a pharmaceutically acceptable salt thereof at a daily dose of about 1.2 mg laquinimod so as to thereby decrease the severity or duration of the relapse in the relapsing-remitting multiple sclerosis human patient.

In an embodiment the administration of the laquinimod increased the odds of the patient to be relapse-free. In another embodiment, the patient receiving laquinimod had approximately 55% better odds to be relapse-free, compared to a patient not receiving the laquinimod treatment.

In further embodiments of the invention, the patient's annualized relapse rate for the first year of treatment is reduced, compared to a patient not receiving the laquinimod treatment. In one embodiment, the reduction is by at least 20%.

In an embodiment, the risk of the patient experiencing a relapse severe enough to require hospitalization is reduced, compared to a patient not receiving the laquinimod treatment. In another embodiment, the risk is reduced by at least 20% or at least 30%. In another embodiment, the risk of the patient experiencing a relapse severe enough to require IV-steroids treatment is reduced, compared to a patient not receiving the laquinimod treatment. In another embodiment, the risk is reduced by at least 20% or at least 30%, compared to a patient not receiving the laquinimod treatment.

The subject invention also provides a method for improving quality of life and general health of a relapsing-remitting multiple sclerosis human patient, the method comprising orally administering to the patient laquinimod or a pharmaceutically acceptable salt thereof at a daily dose of about 1.2 mg laquinimod so as to thereby improve quality of life and general health of the patient.

In a further embodiment of the invention, oral administration of laquinimod or a pharmaceutically acceptable salt thereof to the relapse-remitting multiple sclerosis human patient at a daily dose of about 1.2 mg laquinimod improves the odds of the patient being free of disease or disease activity. In one embodiment, the patient's odds of being disease free is increased by at least 50% or at least 55%, compared to a patient not receiving the laquinimod treatment. In another embodiment, the patient's odds of being free of disease activity is increased by at least 40% or at least 45%, compared to a patient not receiving the laquinimod treatment.

In one embodiment, the method comprises orally administering to the patient laquinimod or a pharmaceutically acceptable salt thereof at a daily dose of substantially 1.2 mg laquinimod. In another embodiment, the method comprises orally administering to the patient laquinimod or a pharmaceutically acceptable salt thereof at a daily dose of 1.2 mg laquinimod. In another embodiment, the laquinimod is administered in the form of laquinimod sodium.

In an embodiment, the laquinimod or pharmaceutically acceptable salt thereof is administered in the form of a tablet. In another embodiment, the laquinimod or pharmaceutically acceptable salt thereof is administered in the form of a capsule.

In an embodiment, the efficacy of laquinimod is measured as compared to a patient not receiving the laquinimod treatment. In another embodiment, the efficacy of laquinimod is measured as compared to the patient at the start of the laquinimod treatment.

The subject invention also provides a pharmaceutical oral unit dosage form of about 1.2 mg laquinimod or a pharmaceutically acceptable salt thereof and a pharmaceutically acceptable carrier for use in reducing the likelihood that the relapsing-remitting multiple sclerosis human patient would experience a confirmed relapse within a predetermined time period, for reducing the severity or duration of a relapse in the relapsing-remitting multiple sclerosis human patient, for improving quality of life and general health of a relapsing-remitting multiple sclerosis human patient, or for improving the odds of a relapsing-remitting multiple sclerosis human patient for being free of disease or disease activity. In one embodiment, the pharmaceutical oral unit dosage form contains substantially 1.2 mg laquinimod. In another embodiment, the pharmaceutical oral unit dosage form contains 1.2 mg laquinimod.

In an embodiment, the pharmaceutical oral unit dosage form is in the form of a tablet. In another embodiment, the pharmaceutical oral unit dosage form is in the form of a capsule.

For the foregoing embodiments, each embodiment disclosed herein is contemplated as being applicable to each of the other disclosed embodiment.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. 2005/0192315 and PCT International Application Publication No. WO 2005/074899, which are hereby incorporated by reference into this application.

A dosage unit may comprise a single compound or mixtures of compounds thereof. A dosage unit can be prepared for oral dosage forms, such as tablets, capsules, pills, powders, and granules.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents flow-inducing agents, and melting agents.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and 2007/146248. These references in their entireties are hereby incorporated by reference into this application.

General techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

### Terms

As used herein, and unless stated otherwise, each of the following terms shall have the definition set forth below.

“Laquinimod” means laquinimod acid or a pharmaceutically acceptable salt thereof. A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention.

“About” in the context of a numerical value or range means±10% of the numerical value or range recited or claimed. “Substantially” in the context of a numerical value or range means±5% of the numerical value or range recited or claimed.

A “dose of 1.2 mg laquinimod” means the amount of laquinimod acid in a preparation is 1.2 mg, regardless of the form of the preparation. Thus, when in the form of a salt, e.g. a laquinimod sodium salt, the weight of the salt form necessary to provide a dose of 1.2 mg laquinimod would be greater than 1.2 mg due to the presence of the additional salt ion.

“Administering to the subject” means the giving of, dispensing of, or application of medicines, drugs, or remedies to a subject to relieve, cure, or reduce the symptoms associated with a disease, disorder or condition.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat multiple sclerosis. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, to “treat” or “treating” encompasses, e.g., inducing inhibition, regression, or stasis of, or ameliorating or alleviating a symptom of, a disease and/or condition. As used herein, “inhibition” of disease progression or complication in a subject means preventing or reducing the disease progression and/or complication in the subject. “Ameliorating” or “alleviating” a condition or state as used herein shall mean to relieve or lessen the symptoms of that condition or state. In addition, to “treat” or “treating” as used herein refers to the periodic administration of a substance, i.e., laquinimod, for a period of at least one month and specifically excludes periodic administration of less than one month.

“Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

As used herein “afflicted”, as in a patient afflicted with a disease or a condition, means a patient who has been affirmatively diagnosed to have the disease or condition. For example, a patient afflicted with multiple sclerosis means a patient who has been affirmatively diagnosed to have multiple sclerosis. The diagnosis of the disease or condition can be effected using any of the appropriate methods known in the art. For multiple sclerosis, the diagnosis is as defined by the Revised McDonald criteria (Polman, 2011). Thus, in an embodiment of the present invention the method includes the step of determining whether a patient is a multiple sclerosis patient.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CDB⁺ T cells, anti-NF-L, anti-CSF 114(G1c)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapsing-Remitting Multiple Sclerosis” or “RRMS” is characterized by clearly defined acute attacks with full recovery or with sequelae and residual deficit upon recovery, where periods between disease relapses are characterized by a lack of disease progression. (Lublin, 1996)

“Confirmed Relapse” is defined as the appearance of one or more new neurological abnormalities or the reappearance or worsening of one or more previously observed neurological abnormalities wherein the change in clinical state lasts at least 48 hours and is immediately preceded by an improving neurological state of at least thirty (30) days from onset of previous relapse. This criterion is different from the clinical definition of relapse which requires only 24 hours duration of symptoms. (EMEA Guideline, 2006) Since “in study” relapse definition must be supported by an objective neurological evaluation as discussed below, a neurological deficit must sustain long enough to eliminate pseudo-relapses.

An event is a relapse only when the subject's symptoms are accompanied by observed objective neurological changes, consistent with at least one of the following: an increase of at least 0.5 in the EDSS score as compared to the previous evaluation, an increase of one grade in the score of 2 or more of the 7 FS functions as compared to the previous evaluation, or an increase of 2 grades in the score of one FS as compared to the previous evaluation.

In addition, the subject must not be undergoing any acute metabolic changes such as fever or other medical abnormality. A change in bowel/bladder function or in cognitive function must not be entirely responsible for the changes in EDSS or FS scores.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS). (Kurtzke J F, 1983)

A “confirmed progression” of BOSS, or “confirmed disease progression” as measured by EDSS score is defined as an increase in EDSS of ≧1 point from baseline for subjects with baseline EDSS of ≦5.0, or an increase in EDSS of ≧0.5 points from baseline for subjects with baseline EDSS of 5.5. In order to be considered a confirmed progression, the increase must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Symbol Digit Modalities Test” or “SDMT” is a measure of cognitive function using a five minute assessment that quickly screens for cerebral dysfunction by means of a simple substitution task. The SDMT is described in, e.g., Smith, 1982; Christodoulou, 2003; Benedict, 2004; Benedict 2005; Benedict 2006; Houtchens, 2007; Benedict 2007; Warlop 2009; and Toledo, 2008.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen. (Mehta, 1996; Grossman, 1994)

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism. (Golder, 2007)

“Modified Fatigue Impact Scale” or “MFIS” is a validated specific subject-reported outcome measure developed to evaluate the impact of fatigue on the lives of people with MS. This instrument provides an assessment of the effects of fatigue in terms of physical, cognitive, and psychosocial functioning. The full-length MFIS consists of 21 items while the abbreviated version has 5 items. (Fisk et al, 1994)

“MS Functional Composite” or “MSFC” is a clinical outcome measure for MS. The MSFC comprises quantitative functional measures of three key clinical dimensions of MS: leg function/ambulation, arm/hand function, and cognitive function. Scores on component measures are converted to standard scores (z-scores), which are averaged to form a single MSFC score. (Fischer, 1999)

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “20-60%” includes 20.0%, 20.1%, 20.2%, 20.3%, 20.4% etc. up to 60.0%.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### ALLEGRO and BRAVO Clinical Trials (Phase III)

ALLEGRO and BRAVO are two clinical trials reported in, e.g., PCT International Application Publication No. WO/2010/147665 (Tarcic et al.).

ALLEGRO was a study performed in subjects with RRMS to assess the efficacy, safety and tolerability of laquinimod 0.6 mg over placebo in a double-blind design. The treatment duration in this study was 24 months and it enrolled 1,106 patients equally distributed between laquinimod 0.6 mg and placebo arms.

The primary endpoint was annualized relapse rate (ARR). Secondary endpoints were gadolinium-enhancing (GdE)-T1 and new-T2 lesions, time to Expanded Disability Status Scale (EDSS) progression confirmed at 3 months and multiple sclerosis functional composite (MSFC) z-score. In ALLEGRO the primary endpoint (ARR) and three key secondary endpoints were met.

Laquinimod treatment effects on the different endpoints are summarized in Table 1 below.

TABLE 1
ALLEGRO: Summary of Efficacy Results

| End-Points | % reduction (p-value) |
|---|---|
| ARR | 23% (0.0024) |
| Brain Atrophy (exploratory endpoint) | 32.8% (<0.0001) |
| EDSS Progression (3m confirmation) | 36% (0.0122) |
| Cumulative number of GdE T1 lesions | 37% (0.0003) |
| Cumulative number of new T2 lesions | 30% (0.0002) |
| MSFC z-score | 51% (0.59) |

BRAVO was a study performed in subjects with RRMS to assess the efficacy, safety and tolerability of laquinimod 0.6 mg over placebo in a double-blind design with a reference arm of IFN-β-1a (Avonex®) in a rater-blinded assessment. The study had treatment duration of 24 months and enrolled 1,331 subjects equally distributed between the three (3) treatment arms. The primary endpoint was ARR. Secondary endpoints were Brain atrophy, time to EDSS progression confirmed at 3 months and MSFC z-score.

The BRAVO study did not meet its primary endpoint. The results showed a reduction in the ARR of 17.7% (p=0.0746) in laquinimod treated patients as compared to placebo. One of the basic assumptions used to assess the sample size for the study was that treatment with laquinimod will reduce the patient population ARR by 25% or more when compared to the placebo group. Thus, the BRAVO study was not powered to detect a statistically significant reduction of 17.7%.

The comparator, Avonex® showed a reduction of 25.9% (p=0.0067). Although no deficiencies were found within the randomization process, review of baseline characteristics revealed differences between the laquinimod and placebo arms in two baseline magnetic resonance imaging (MRI) findings (percent of patients with GdE-T1 lesions and mean volume of T2 lesions (cm3)). In light of this baseline imbalance, these two baseline MRI parameters were added to the model as additional covariates. Using this corrected post-hoc analysis, the primary endpoint of the BRAVO study showed very similar results to those obtained in the ALLEGRO study in that laquinimod reduced the ARR by 21.3% (p=0.0264). The comparator, Avonex®, showed a 28.6% reduction in ARR (p=0.0021) compared to placebo after the correction. It is inventors' assessment that the corrected results more adequately represent the true treatment effect of laquinimod.

The treatment effects of laquinimod and the comparator Avonex® on the different endpoints are summarized in Table 2 below:

TABLE 2
BRAVO: Summary of Efficacy Results

| | Laquinimod 0.6 mg | | Avonex ® | |
|---|---|---|---|---|
| | Original | Corrected | Original | Corrected |
| | % reduction | % reduction | % reduction | % reduction |
| End-Points | (p-value) | (p-value) | (p-value) | (p-value) |
| ARR | 17.7% | 21.3% | 25.9% | 28.7% |
| | (0.0746) | (0.0264) | (0.0067) | (0.0021) |
| Brain Atrophy | 27.6% | 27.4% | −10% | −9% |
| | (0.0001) | (<0.0001) | (0.14) | (0.14) |
| EDSS Progression (3m | 31.3% | 33.5% | 25.8% | 28.7% |
| confirmation) | (0.06) | (0.04) | (0.13) | (0.09) |
| MSFC | 77.7 | 77.1% | 66.6 | 63.7% |
| | (0.1505) | (0.1152) | (0.2083) | (0.1582) |
| Cumulative number of GdE | 21.5% | 21.7% | 61.5% | 60% |
| T1^{a} lesions | (0.07) | (0.062) | (<0.0001) | (<0.0001) |
| Cumulative number of new | 16.5% | 18.7% | 51.1% | 52.3% |
| T2 lesions (exploratory | (0.08) | (0.037) | (<0.0001) | (<0.0001) |
| endpoint) | | | | |

### Example 2

### Clinical Trial (Phase III)—Assessment of Oral Laquinimod in Preventing Progression of MS

A multinational, multicenter, randomized, double-blind, parallel-group, placebo-controlled study followed by an active treatment (clinical trial MS-LAQ-305) is conducted to evaluate the efficacy, safety and tolerability of two doses of oral administration of laquinimod (0.6 mg/day or 1.2 mg/day) laquinimod in subjects with relapsing remitting multiple sclerosis (RRMS).

### Study Duration

Screening period: up to 1 month.
Double-blind Placebo-controlled (DBPC) period (Period 1): At least 15 months, but not more than 24 months of once-daily, oral administration of either laquinimod 0.6 mg, 1.2 mg or matching oral placebo. DBPC period for all subjects is declared closed when all ongoing enrolled subjects complete at least 15 months of treatment.
Active-treatment (AT) period (Period 2): In this period (24 months), subjects who are assigned to either 0.6 mg or 1.2 mg daily oral laquinimod during the DBPC period continue with the same treatment assignment, whereas those who are assigned to placebo receive 1.2 mg daily oral laquinimod.

### Study Population

Subjects with Relapsing Remitting Multiple Sclerosis (RRMS).

### Study Design

Eligible subjects (approximately 1,800) are randomized in a 1:1:1 ratio into one of the following treatment arms:
1. Laquinimod 0.6 mg: two capsules, one containing 0.6 mg laquinimod and the other containing matching placebo, administered orally once daily.
2. Laquinimod 1.2 mg: two capsules containing 0.6 mg laquinimod administered orally once daily.
3. Matching placebo: two capsules containing placebo (matching to the 0.6 mg) administered orally once daily.

The study comprises of two treatment periods, Double-blind Placebo-controlled (DBPC) and Active-treatment (AT). Subjects who complete 24 months on study drug in Period 1 or complete at least 15 months on study drug when Period 1 is declared closed continue on to Period 2.

During Period 1, subjects are evaluated at study sites at months: −1 (screening), 0 (baseline), 1, 2, 3 and every 3 months thereafter until completion visit of Period 1.

When Period 1 is declared closed, subjects who complete at least 15 months in the study are requested to attend a completion visit of Period 1. Completion activities that have already been performed are not repeated for subjects who completed a visit within the month prior to this visit.

Subjects who stopped treatment with the study drug before the completion visit of Period 1 are considered Early Treatment Discontinuation (ETD) subjects. During Period 1 ETD subjects continue follow up according to scheduled visits (until completion visit of Period 1). Subjects that do not complete follow up, for any reason, are considered Early Study Discontinuation (ESD) subjects.

The completion visit of Period 1 serves as the baseline visit of Period 2. During Period 2, subjects are evaluated at study sites at months 0 AT (baseline, completion visit of Period 1), 1 AT, 2 AT, 3 AT and every 3 months thereafter until completion/ETD of Period 2. Subjects who are ETD during period 2 are followed only if indicated for resolution of AE or relapse.

The following assessments are performed at the specified time points:
1. Vital signs are measured at each study visit.
2. A physical examination is performed at months −1 (screening), 0 (baseline) 1, 3, 6, and every 6 months thereafter, ETD (if applicable) and until completion visit of Period 1. During Period 2, a physical examination is performed at months 0 AT (baseline, completion visit of Period 1), 1 AT, 3 AT, 6 AT and every 6 months thereafter, until completion/ETD of Period 2.
3. The following safety clinical laboratory tests are performed:
   (a) Complete blood count (CBC) with differential—at all scheduled visits during Periods 1 and 2.
   (b) Serum chemistry (including electrolytes, liver enzymes, urea, creatinine, calculated Glomerular Filtration Rate (GFR)—at screening and prior to each MRI scan, glucose, total protein, albumin, direct and total bilirubin and pancreatic amylase)—at all scheduled visits during the DBPC and AT periods. Calculated Glomerular Filtration Rate (GFR) is done at screening and prior to each MRI scan in both study periods.
   (c) Lipid profile (total cholesterol, HDL, LDL, triglycerides)—at baseline and every 12 months during the DBPC and AT periods.
   (d) Urinalysis—at the screening visit.
   (e) Serum β-hCG (human choriogonadotropin beta) in women of child-bearing potential—at each scheduled study visit during the DBPC and AT periods.
   (f) Urine β-hCG test in women of child-bearing potential—at baseline (month 0) and at all scheduled visits during the DBPC and AT periods.
   (g) Starting after visit Month 3, between scheduled visits, a rapid urine β-hCG test is performed in women of child-bearing potential every 28 (±2) days. The subject is contacted within 72 hours after the scheduled test is performed and asked specific questions regarding the test. In case of suspected pregnancy (positive urine β-hCG test result) the caller instructs the subject to make sure that the study drug has been discontinued and the subject arrives at the site as soon as possible (within 10 days) with all study drugs—during the DBPC and AT periods.
4. ECG is performed at months −1 (screening), 0 (baseline, three recordings 10 min apart, before first dose), 1, 2, 3, 6, and every 6 months thereafter until completion visit of Period 1 and ETD visit (if applicable). During Period 2, ECG is performed at months 0 AT (baseline, completion visit of Period 1), 1 AT, 2 AT, 3 AT, 6 AT and every 6 months thereafter until completion/ETD of Period 2.
5. Chest X-ray is performed at months −1 (screening), (if not performed within 6 months prior to the screening visit).
6. Adverse Events (AEs) are monitored throughout the study during Periods 1 and 2.
7. Concomitant Medications are monitored throughout the study—during Periods 1 and 2.
8. The subjects undergo MRI scans at months 0 (baseline) and 15 and an additional MRI is performed at ETD visit (if applicable) and completion visit of Period 1, provided no MRI was performed within the previous 3 months. During Period 2, MRI is performed at months 0 AT (baseline, completion visit of Period 1) and completion/ETD of Period 2. In cases of ETD, the additional MRI is performed, provided no MRI was performed within the previous 3 months.
9. Neurological evaluations, including Expanded Disability Status Scale (EDSS), Functional Systems (FS) and Timed 25-foot walk (T25FW) are performed at months −1 (screening (excluding T25FW)), 0 (baseline) and every 3 months thereafter, ETD visit (if applicable) and until completion visit of Period 1. During Period 2, neurological evaluations, including EDSS, FS and T25FW are performed at months 0 AT (baseline, completion visit of Period 1) and every 3 months thereafter until completion/ETD of Period 2.
10. Symbol Digit Modalities Test (SDMT) is performed at months 0 (baseline), 6, 12, 15, 24, ETD visit (if applicable) and completion visit of Period 1. During Period 2, SDMT is performed at months 0 AT (baseline; completion visit of Period 1) and every 6 months thereafter, until completion/ETD of Period 2.
11. The general health status is assessed by the EuroQoL (EQ-5D) questionnaire at months 0 (baseline), ETD visit (if applicable) and completion visit of Period 1. During Period 2, EQ-5D is performed at months 0 AT (baseline; completion visit of Period 1) and completion/ETD of Period 2.
12. The general health status will be assessed by the Short-Form general health survey (SF-36) subject-reported questionnaire at months 0 (baseline) and every 6 months thereafter, ETD visit (if applicable) and until completion visit of Period 1. During Period 2, SF-36 is performed at months 0 AT (baseline; completion visit of Period 1) and every 6 months thereafter, until completion/ETD of Period 2.
13. Pharmacokinetic (PK) study: Blood samples for analysis of laquinimod plasma concentrations are collected from all subjects at Months 1, 6 and 12 of Period 1.
14. Relapses are confirmed/monitored throughout the study.

### Relapse Treatment

The allowed treatment for a relapse is intravenous Methylprednisolone 1 gr/day for up to 5 consecutive days.

### Re-Consent Criteria

During Period 1, subjects that meet either of the following criteria are reminded of the current available MS medications and the opportunity to terminate the study and are requested to re-sign a designated informed consent form if he/she chooses to continue to participate in the study, in the same treatment assignment:
The subject experiences a confirmed multiple sclerosis (MS) relapse, (as defined in the protocol).
The subject experiences Confirmed Disease Progression (CDP), defined as an increase in EDSS of 1 point from baseline for subjects with baseline EDSS of 55.0, or an increase in EDSS of 0.5 points from baseline for subjects with baseline EDSS of 5.5. This increase should be sustained for at least 3 months. Progression cannot be confirmed during a relapse.

Subjects that do not sign the re-consent form discontinue treatment with the study drug (ETD) and continue follow up according to scheduled visits of Period 1 (until completion visit of Period 1).

### Ancillary Studies:

Pharmacogenetic (PGx) assessment: Blood samples for PGx parameters are collected from all subjects that signed the informed consent form during the DBPC period, preferably at month 0 (baseline DBPC period) or any other visit following month 0 during Period 1.
Whole blood and serum samples (in selected countries and sites) are collected for evaluation of the immunological response to treatment with laquinimod and further investigation of the potential mechanism of action, at months: 0, 1, 3, and 12 of Period 1.
Magnetization Transfer (MT) (in selected countries and sites) is assessed at months 0 (baseline) and 15. An additional MRI is performed at completion visit of Period 1 and ETD visit (if applicable), provided no MRI was performed within the previous 3 months.
3D T1-w acquisition of the cervical cord (in selected countries and sites) is assessed at months 0 (baseline) and 15. An additional MRI is performed at completion visit of Period 1 and ETD visit (if applicable), provided no MRI was performed within the previous 3 months.

### Inclusion/Exclusion Criteria

### Inclusion Criteria

1. Subjects must have a confirmed and documented MS diagnosis as defined by the Revised McDonald criteria (Dolman, 2011) with relapse onset disease or a relapsing-remitting disease course.
2. Subjects must be ambulatory with Kurtzke EDSS score of 0-5.5 in both screening and randomization visits.
3. Subjects must be in a stable neurological condition, relapse-free and free of any corticosteroid treatment [intravenous (IV), intramuscular (IM) and/or per os (PO)] or adrenocorticotrophic hormone (ACTH), 60 days prior to randomization.
4. Subjects must have experienced at least one documented relapse in the 12 months prior to randomization.
5. Subjects must be between 18 and 55 years of age at screening, inclusive.
6. Subjects must have disease duration of at least 6 months, but not more than 12 years (from the first symptom) prior to randomization.
7. Women of child-bearing potential must practice an acceptable method of birth control until 30 days after the last dose of treatment was administered [acceptable methods of birth control in this study include: surgical sterilization, intrauterine devices, oral contraceptive, contraceptive patch, long-acting injectable contraceptive or double-barrier method (condom or diaphragm with spermicide)].
8. Subjects must be able to sign and date a written informed consent prior to entering the study.
9. Subjects must be willing and able to comply with the protocol requirements for the duration of the study.

### Exclusion Criteria

1. Subjects with progressive forms of MS.
2. Subjects with Neuromyelitis Optica (NMO).
3. Use of experimental or investigational drugs (including dimethyl fumarate and Teriflunomide) and/or participation in drug clinical studies within 6 months prior to randomization.
4. Use of immunosuppressive agents, including fingolimod (Gilenya®) or cytotoxic agents, including Cyclophosphamide within 6 months prior to randomization.
5. Use of any of the following within 2 years prior to randomization: natalizumab (Tysabri®), rituximab, ocrelizumab, atacicept, belimumab, or ofatumumab.
6. Previous treatment with glatiramer acetate (Copaxone®) Interferon-β (either 1a or 1b) or intravenous immunoglobulin (IVIG) within 2 months prior to randomization.
7. Chronic (more than 30 consecutive days) systemic (IV, IM or PO) corticosteroid treatment within 2 months prior to randomization.
8. Previous use of Mitoxantrone (Novantrone®), Cladribine, or alemtuzumab (CAMPATH-1H).
9. Previous use of laquinimod.
10. Previous total body irradiation or total lymphoid irradiation.
11. Previous stem cell treatment, autologous bone marrow transplantation or allogenic bone marrow transplantation.
12. Use of moderate/strong inhibitors of CYP3A4 within 2 weeks prior to randomization.
13. Use of inducers of CYP3A4 within 2 weeks prior to randomization.
14. Pregnancy or breastfeeding.
15. Serum levels ≧3×ULN of either ALT or AST at screening.
16. Serum direct bilirubin which is ≧2×ULN at screening.
17. Subjects with a clinically significant or unstable medical or surgical condition that would preclude safe and complete study participation, as determined by medical history, physical examinations, ECG, laboratory tests MRI or chest X-ray. Such conditions may include:
   A cardiovascular or pulmonary disorder that cannot be well-controlled by allowed medications permitted by the study protocol.
   A Central Nervous System (CNS) disorder other than MS that may jeopardize the subject's participation in the study, including such disorders that are demonstrated on the baseline MRI.
   A gastrointestinal disorder that may affect the absorption of study medication.
   Renal disease.
   Any form of acute or chronic liver disease.
   Known human immunodeficiency virus positive status.
   A history of drug and/or alcohol abuse.
   Unstable psychiatric disorder.
   Any malignancies, excluding basal cell carcinoma, in the 5 years prior to randomization.
18. A known history of sensitivity to gadolinium (Gd).
19. GFR≦60 mL/min at the screening visit.
20. Inability to successfully undergo MRI scanning.
21. Subjects who underwent endovascular treatment for Chronic Cerebrospinal Venous Insufficiency (CCSVI) within 3 months prior to randomization.
22. Known hypersensitivity that would preclude administration of laquinimod capsule, such as hypersensitivity to mannitol, meglumine or sodium stearyl fumarate.

### Outcome Measures

### Primary Outcome Measure

Time to Confirmed Disease Progression (CDP) during the DBPC period, where CDP is defined as an increase in EDSS of ≧1 point from baseline for subjects with baseline EDSS of ≦5.0, or an increase in EDSS of ≧0.5 points from baseline for subjects with baseline EDSS of 5.5. This increase should be sustained for at least 3 months. Progression cannot be confirmed during a relapse.

Analysis is performed at the completion of the DBPC period.

### Secondary Outcome Measures

Brain atrophy as defined by the percent change in brain volume from baseline to month 15 (for subjects that performed ETD, the MRI from the ETD visit is included in the analysis provided the subject completed 9 months or treatment).
The time to first confirmed relapse during the DBPC period.

### Safety and Tolerability Outcome Measures

1. Adverse events
2. Vital signs
3. ECG findings
4. Clinical laboratory parameters
5. Proportion of subjects (%) who prematurely discontinued from the study, reason of discontinuation and the time to ETD.
6. Proportion of subjects (%) who prematurely discontinued from the study due to AEs and the time to withdrawal.

### Additional Exploratory Endpoints

Exploratory endpoints include Cognitive (SDMT), MRI and quality of life. MRI endpoints are analyzed based on scans performed at month 15 and 24. Exploratory endpoints include:
Change from baseline in the Symbol Digit Modalities Test (SDMT) score.
Annualized Relapse Rate (ARR).
Brain atrophy as defined by the percent change in brain volume from baseline to month 24.
Number of GdE-T1 lesions.
Number of new T2 lesions.
Number of new T1 hypointense lesions (black holes).
Change from baseline in T2 lesions volume.
Change from baseline in GdE-T1 lesions volume.
Change from baseline in T1 hypointense lesions volume (black holes).
The general health status, as assessed by the EuroQoL (EQ-5D) questionnaire.
The general health status and health-related quality of life, as assessed by the Short-Form general health survey (SF-36) subject-reported questionnaire.
Change from baseline in disability as assessed by the Timed 25-foot walk (T25FW).

### Primary End Point Analysis

The primary endpoint of the study is the time to CDP during Period 1. The primary analysis for the comparisons between each dose of laquinimod (0.6 mg and 1.2 mg) vs. placebo is conducted utilizing the baseline adjusted Cox's proportional hazards (PH) model (SAS® PROC PHREG). Categorical EDSS at baseline (≦4 or >4), Country/Geographical Region (CGR), categorical age at baseline (≦40 or >40) and T2 volume at baseline are included as covariates in the model. In addition, the time to confirmed progression of EDSS is presented by Kaplan-Meier curves stratified by treatment group. The adequacy of the proportional hazards assumption is confirmed by including two time dependent covariates of dose by log (time) interactions in the primary analysis model and testing each of them in 5% level. In case the PH assumption is rejected for a certain dose, the log rank test (SAS® PROC LIFTEST) is used for statistical inference in this dose.

### Secondary End-Points Analyses

The analysis of brain atrophy as measured by Percent Brain Volume Change from baseline (PBVC) to month 15 is based on two contrasts between laquinimod 0.6 mg and 1.2 mg vs. placebo while utilizing the baseline-adjusted analysis of covariance (SAS® PROC GLM). In addition to treatment group, normalized brain volume at baseline, Indicator of GdE lesions at baseline (≧1 vs. 0), T2 volume at baseline and CGR are used as covariates.

The analysis of the time to confirmed relapse during Period 1 is based on two contrasts between laquinimod 0.6 mg and 1.2 mg vs. placebo utilizing the baseline adjusted Cox's proportional hazards model Regression (SAS® PROC PHREG). In addition to treatment group, baseline EDSS score, log of the prior 2-year relapses (+1), CGR, Indicator of GdE lesions at baseline (≧1 vs. 0) and T2 volume are used as covariates. The adequacy of the proportional hazards assumption is confirmed by including two time-dependent covariates of dose by log (time) interactions in the primary analysis model and testing each of them in 5% level.

### Results

This clinical study shows that, as compared to 0.6 mg/day laquinimod treatment, 1.2 mg/day laquinimod treatment show improved efficacy in treating RRMS patients with respect to all endpoints. Specifically, as compared to 0.6 mg/day laquinimod treatment, 1.2 mg/day laquinimod treatment is more effective in shortening the time to CDP and time to confirmed relapse, reducing brain atrophy, as measured by percent brain volume change from baseline, reducing relapse rate, slowing the progression of disability, and reducing the development of new MRI lesions in RRMS patients.

According to the study, as compared to RRMS patients treated daily oral administration of 0.6 mg laquinimod or placebo, RRMS patients treated with daily oral administration of 1.2 mg laquinimod experience a prolonged time to CDP. In addition, as compared to RRMS patients treated daily oral administration of 0.6 mg laquinimod or placebo, RRMS patients treated with daily oral administration of 1.2 mg laquinimod have reduced brain atrophy, as measured by percent brain volume change from baseline to month 15. Moreover, as compared to patients treated with daily oral administration of 0.6 mg laquinimod and placebo, patients treated with daily oral administration of 1.2 mg laquinimod experience a prolonged time to first confirmed relapse. Furthermore, as compared to RRMS patients treated daily oral administration of 0.6 mg laquinimod or placebo, RRMS patients treated with daily oral administration of 1.2 mg laquinimod have reduced number of confirmed relapses, which is directly related to the relapse rate.

Still further, as compared to RRMS patients treated daily oral administration of 0.6 mg laquinimod or placebo, RRMS patients treated with daily oral administration of 1.2 mg laquinimod have improved Symbol Digit Modalities Test (SDMT) score, lower annualized relapse relate, reduced brain atrophy as measured by the percent change in brain volume from baseline to month 24, reduced the accumulation of disability as measured by the MSFC score or Timed 25-foot walk (T25FW), reduced MRI-monitored disease activity in RRMS patients, as measured by the cumulative number of enhancing lesions on T₁-weighted images, the cumulative number of new hypointense lesions on T₁-scans, the cumulative number of new T₂ lesions, number of GdE-T1 lesions, number of new T2 lesions, number of new T1 hypointense lesions (black holes), change from baseline in T2 lesions volume, change from baseline in GdE-T1 lesions volume, and change or change from baseline in T1 hypointense lesions volume (black holes).

Yet further, fatigue and functional status of patients treated with 1.2 mg/day laquinimod was maintained or improved as compared to patients treated with 0.6 mg/day laquinimod or placebo. Finally, as compared to RRMS patients treated daily oral administration of 0.6 mg laquinimod or placebo, RRMS patients treated with daily oral administration of 1.2 mg laquinimod experiences show improved functional status and general health, as assessed by the Short-Form general health survey (SF-36) subjected-reported questionnaire.

Finally, daily oral administration of 1.2 mg laquinimod is more effective in providing neuroprotection to the patients as compared to daily oral administration of 0.6 mg laquinimod or placebo.

### REFERENCES

1. PCT International Application Publication No. WO 2007/047863, published Apr. 26, 2007, international filing date Oct. 18, 2006.
2. PCT International Application Publication No. WO 2007/146248, published Dec. 21, 2007, international filing date Jun. 12, 2007.
3. PCT International Application Publication No. WO 2010/147665, published Dec. 23, 2010 (Tarcic at al.).
4 Barkhof, F. (1999) “MRI in Multiple Sclerosis: Correlation with Expanded Disability Status Scale (EDSS)”, Multiple Sclerosis. 5(4):283-286 (Abstract).
5. Benedict et al. (2004) “Correlating brain atrophy with cognitive dysfunction, mood disturbances, and personality disorder in multiple sclerosis”. J Neuroimaging; 14(3 Suppl):36S-45S.
6. Benedict et al. (2004) “Prediction of neuropsychological impairment in multiple sclerosis: Comparison of conventional magnetic resonance imaging measures of atrophy and lesion burden” Archiv Neurol; 61:226-230.
7. Benedict et al. (2005) “Regional lobar atrophy predicts memory impairment in multiple sclerosis”. Am J Neuroradiol; 26:1824-1831.
8. Benedict at al. (2007) “Diffusion-weighted imaging predicts cognitive impairment in multiple sclerosis”. Mult Scler; 13(6):722-730.
9. Benedict R H B and Zivadinov R. (2006) “Predicting neuropsychological abnormalities in multiple sclerosis”. J Neurol Sci; 245:67-72.
10. Bjartmar and Fox (2002) “Pathological mechanisms and disease progression of multiple sclerosis: therapeutic implication”, Drugs of Today. 38:7-29.
11. Brex et al. (2002) “A longitudinal study of abnormalities on MRI and disability from multiple sclerosis”, N Engl J Med. Jan. 17, 2002 346(3):158-64.
12. Bruck W and Wegner C. (2011) “Insight into the mechanism of laquinimod action”. J Neurol Sci; 306(1-2):173-79.
13. Bruck W and Zamvill SS. (2012) “Laquinimod, a once daily oral drug in development for the treatment of relapsing remitting multiple sclerosis”. Exp Rev Clin Pharmacol; in press.
14. Brunmark et al. (2002) “The new orally active immunoregulator laquinimod (ABR-215062) effectively inhibits development and relapses of experimental autoimmune encephalomyelitis”, J Neuroimmunology. 130:163-172.
15. Christodoulou et al. (2003) “Cognitive performance and MR markers of cerebral injury in cognitively impaired MS patients”. Neurology; 60:1793-1798.
16. Cohen et al. (2010) “Oral fingolimod or intramuscular interferon for relapsing multiple sclerosis”. N Eng J Med; 362:402-415.
17. Comi at al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; April 28-May 5, 2007; Boston, Mass.
18. Comi et al. (2008) “Effect of laquinimod on MRI-monitored disease activity in patients with relapsing-remitting multiple sclerosis: a multicentre, randomised, double-blind, placebo-controlled phase IIb study”, Lancet. 371:2085-2092.
19. Comi at al. (2009) for the LAQ/5062 Clinical Advisory Board and Study Group. Long-term open extension of oral laquinimod in patients with relapsing multiple sclerosis shows favorable safety and sustained low relapse rate and MRI activity. [Ectrims abstract P443]. Mult Scler. 15(Suppl 2):S127.
20. Comi at al. (2010) for the LAQ/5062 Clinical Advisory Board and Study Group. The effect of laquinimod on MRI-monitored disease activity in patients with relapsing-remitting multiple sclerosis: a double-blind active extension of the multicentre, randomised, double-blind, parallel-group placebo-controlled study. Mult Scler. 16:1360-1366.
21. Cutter et al. (1999) “Development of a multiple sclerosis functional composite as a clinical trial outcome measure”, Brain. 122:871-882.
22. De Stefano et al. (1999) “Evidence of early axonal damage in patients with multiple sclerosis”, Neurology. 52(Suppl 2):A378.
23. Drake et al. (2008) “The Symbol-Digit Modalities Test more accurately reflects multiple sclerosis patient-perceived cognitive impairment compared to the Paced Auditory Serial Addition Test (abstract)”. Mult Scler; 14:S225-S256.
24. Dunitz. M. (1999) Multiple sclerosis therapeutics, Ed. Rudick and Goodkin. London: Taylor & Francis, 1999.
25. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
26. EMEA Guideline on Clinical Investigation of Medicinal Products for the Treatment of Multiple Sclerosis (CPMP/EWP/561/98 Rev. 1, Nove. 2006).
27. EPAR, Rebif®, Scientific Discussion.
28. Fischer et al. (1999) “The Multiple Sclerosis Functional Composite measure (MSFC): an integrated approach to MS clinical outcome assessment” Multiple Sclerosis. 5(4):244-250.
29. Fisk et al. (1994) “Measuring the Functional Impact of Fatigue: Initial Validation of Fatigue Impact Scale”, Clin Inf Dis. 18 Suppl 1:579-83.
30. Fisk at al. (1994) “The Impact of Fatigue on Patients with Multiple Sclerosis”, Can J Neurol Sci. 21:9-14.
31. Frohman et al. (2003) “The utility of MRI in suspected MS: report of the Therapeutics and Technology Assessment Subcommittee of the American Academy of Neurology”, Neurology. Sep. 9, 2003, 61(5):602-11.
32. Giovannoni et al. (2010) “A placebo-controlled trial of oral cladribine for relapsing multiple sclerosis”, N Eng J Med. 362:416-426.
33. Golder W. (2007) “Magnetic resonance spectroscopy in clinical oncology”, Onkologie. 27(3): 304-9.
34. Grossman at al. (1994) “Magnetization transfer: theory and clinical applications in neuroradiology”, RadioGraphics. 14:279-290.
35. Gurevich et al. (2001) “Laquinimod suppress antigen presentation in relapsing-remitting multiple sclerosis: In-vitro high-throughput gene expression study”. Journal of Neuroimmunology; 221:87-94.
36. Hartung at al. (2005) “Significance of neutralizing antibodies to interferon beta during treatment of multiple sclerosis: expert opinions based on the Proceedings of an International Consensus Conference”, Eur J Neurol. 12:588-601.
37. Hauser et al. (1983) “Intensive immunosuppression in progressive multiple sclerosis”, New Engl J Med. 308:173-180.
38. Hohlfeld et al. (2000) “The neuroprotective effect of inflammation: implications for the therapy of multiple sclerosis”, J Neuroimmunol. 107:161-166.
39. Houtchens et al. (2007) “Thalamic atrophy and cognition in multiple sclerosis”. Neurology; 69:113-123.
40. Jacobs et al. (1996) “Intramuscular interferon beta-1a for disease progression in relapsing multiple sclerosis”, Ann Neurol. 39:285-294.
41. Kappos et al. (2010) “A placebo-controlled trial of oral fingolimod in relapsing multiple sclerosis”, N Eng J Med. 362:387-401.
42. Kurtzke J F. (1983) “Rating neurologic impairment in multiple sclerosis: an expanded disability status scale (EDSS)”, Neurology, 33(11):1444-1452.
43. Lazeron at al. (2005) “Brain atrophy and lesion load as explaining parameters for cognitive impairment in multiple sclerosis”. Mult Scler; 11:524-531.
44. Lublin F D and Reingold S C. (1996) “Defining the clinical course of multiple sclerosis”. Neurology; 46: 907-11.
45. McDonald at al. (2001) “Recommended diagnostic criteria for multiple sclerosis: guidelines from the International Panel on the diagnosis of multiple sclerosis”. Ann Neurol; 50:121-127.
46. Mehta et al. (1996) “Magnetization transfer magnetic resonance imaging: a clinical review”, Topics in Magnetic Resonance Imaging 8(4):214-30.
47. Miki at al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
48. Miller et al. (2007) “MRI outcomes in a placebo-controlled trial of natalizumab in relapsing MS”, Neurology. 68:1390-1401.
49. Neuhaus at al. (2003) “Immunomodulation in multiple sclerosis: from immunosuppression to neuroprotection”, Trends Pharmacol Sci. 24:131-138.
50. Noonan et al. (2002) “Prevalence estimates for MS in the United States and evidence of an increasing trend for women”. Neurology; 58 (1):136-138.
51. Noonan et al. (2010) “The prevalence of multiple sclerosis in 3 US communities”. Preventing chronic disease; 7 (1):A12.
52. Noseworthy et al. (2000) “Multiple sclerosis”, N Engl J Med. 343:938-952.
53. Noseworthy at al. (2000) “Linomide in relapsing and secondary progressive MS. Part 1: Trial Design and clinical results”, Neurology. 54:1726-1733.
54. Panitch at al. for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
55. Parmenter et al. (2007) “Screening for cognitive impairment in MS using the Symbol Digit Modalities Test”. Mult Scler; 13:52-57.
56. Polman et al. (2005) “Diagnostic criteria for multiple sclerosis: 2005 revisions to the McDonald Criteria”, Annals of Neurology, Volume 58 Issue 6, Pages 840-846.
57. Polman et al. (2005) “Treatment with laquinimod reduces development of active MRI lesions in relapsing MS”, Neurology. 64:987-991.
58. Polman et al. (2006) “A randomized, placebo-controlled trial of natalizumab for relapsing multiple sclerosis”, N Eng J Med. 354:899-910.
59. Polman et al. (2011) “Diagnostic Criteria for Multiple Sclerosis: 2010 Revisions to the “McDonald Criteria”. Ann Neurol; 69:292-302.
60. Poser et al. (1983) “New Diagnostic Criteria for Multiple Sclerosis: Guidelines for Research Protocols”, Annals of Neurology, March 1983, 13(3):227-230.
61. Preiningerova J. (2009) “Oral laquinimod therapy in relapsing multiple sclerosis”, Expert Opin Investig Drugs. 18:985-989.
62. PRISMS Study Group. Randomized double-blind placebo-controlled study of interferon β-1a in relapsing/remitting multiple sclerosis. Lancet 1998; 352:1498-1506.
63. Pugliatti et al. (2006) “The epidemiology of multiple sclerosis in Europe”. European journal of neurology: the official journal of the European Federation of Neurological Societies; 13 (7):700-722.
64. Randolph et al. (2001) “Metamemory and tested cognitive functioning in multiple sclerosis”. Clin Neuropsychol; 15:357-368.
65. Rosen Y. (2007) “The Recent advances in magnetic resonance neurospectroscopy”, Neurotherapeutics. 27(3): 330-45.
66. Rudick et al. (1999) “Use of the brain parenchymal fraction to measure whole brain atrophy in relapsing-remitting MS: Multiple Sclerosis Collaborative Research Group”. Neurology. 53:1698-1704.
67. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
68. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
69. Runstrom et al. (2006) “Inhibition of the development of chronic experimental autoimmune encephalomyelitis by laquinimod (ABR-215062) in IFN-β k.o. and wild type mice.” J Neuroimmunol; 173: 69-78.
70. Sandberg-Wollheim et al. (2005) “48-week open safety study with high-dose oral laquinimod in patients”, Mult Scler. 11:S154 (Abstract).
71. SIENA and SIENAX available from the FMRIB Software Library, Oxford University. Oxford, UK; http://www.fmrib.ox.ac.uk/analysis/research/siena/siena.
72. Smith A. (1982) Symbol Digit Modalities Test: Manual. Los Angeles: Western Psychological Services.
73. Sorenson P S. (2006) “Neutralising antibodies to interferon-β-measurement, clinical relevance, and management”, J Neurol. 253[Suppl 6]:VI/16-VI/22.
74. Sormani et al. (2004) “Measurement error of two different techniques for brain atrophy assessment in multiple sclerosis”, Neurology. 62:1432-1434.
75. Stankiewicz et al. (2009) “Brain MRI lesion load at 1.5 T and 3 T versus clinical status in multiple sclerosis”. J Neuroimaging. doi:10.1111/j.1552-6569.2009.00449.x.
76. Strober L et al. (2009) “Sensitivity of conventional memory tests in multiple sclerosis: comparing the Rao Brief Repeatable Neuropsychological Battery and the Minimal Assessment of Cognitive Function in MS”. Mult Scler; 15:1077-1084.
77. Temple R. (2006) “Hy's law: predicting serious hepatoxicity”, Pharmacoepidemiol Drug Saf. 15(4):241-3.
78. The IFNB Multiple Sclerosis Study Group. (1993) Interferon beta-1b is effective in relapsing-remitting multiple sclerosis. I. Clinical results of a multicenter, randomized, double-bind, placebo-controlled trial. Neurology; 43:655-661.
79. The IFNB Multiple Sclerosis Study Group. (1993) Interferon beta-1b is effective in relapsing-remitting multiple sclerosis. II. MRI analysis results of a multicenter, randomized, double-blind, placebo-controlled trial. Neurology; 43:662-667.
80. The National MS Society (USA), The Disease Modifying Drug Brochure, Oct. 19, 2006.
81. Thone and Gold (2011) “Laquinimod: a promising oral medication for the treatment of relapsing-remitting multiple sclerosis”, Expert Opin Drug Metab Toxicol. 2011 March; 7(3): 365-70.
82. Toledo et al. (2008) “Retinal nerve fiber layer atrophy is associated with physical and cognitive disability in multiple sclerosis”. Mult Scler; 14:906-912.
83. US Food and Drug Administration, Center for Drug Evaluation and Research. Peripheral and Central Nervous System (PCNS) Advisory Committee. US Department of Health and Human Services 2006. Briefing Document. Biogen Idec Biologics Marketing Application STN 125104/15. Natalizumab (Tysabri) for Multiple Sclerosis. Dated Feb. 9, 2006. Pages 45-48.
84. Warlop et al. (2009) “Transverse diffusivity of cerebral parenchyma predicts visual tracking performance in relapsing-remitting multiple sclerosis”. Brain Cogn; 71:410-415.
85. Wegner et al. (2010) “Laquinimod interferes with migratory capacity of T cells and reduces IL-17 levels, inflammatory demyelination and acute axonal damage in mice with experimental autoimmune encephalomyelitis”. J Neuroimmunol; 227(1-2):133-143.
86. Wegner et al. “Laquinimod reduces cuprizone-induced demyelination by down modulation of astrocytic NFkB activation”. Acta Neuorpathologica, submitted.
87. Yang at al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
88. Zou at al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method of treating a human patient afflicted with multiple sclerosis or presenting a clinically isolated syndrome, the method comprising orally administering to the human patient laquinimod or a pharmaceutically acceptable salt thereof at a daily dose of about 1.2 mg laquinimod so as to thereby treat the human patient.

2. The method of claim 1, wherein the administration of laquinimod is effective to alleviate a symptom of or a condition associated with multiple sclerosis.

3. The method of claim 2, wherein the administration of laquinimod is effective to increase the time to confirmed disease progression, increase the time to confirmed relapse, reduce brain atrophy, reduce relapse rate, reduce rate of confirmed relapses requiring hospitalization and/or IV steroids, reduce the accumulation of disability, reduce or inhibit progression of the level of fatigue, improve or inhibit deterioration of the functional status, improve or inhibit deterioration of the general health, reduce MRI-monitored disease activity or reduce cognitive impairment in the human patient.

4. The method of claim 3, wherein the administration of laquinimod is effective to increase the time to confirmed disease progression in the human patient.

5. The method of claim 4, wherein confirmed disease progression is measured by Kurtzke Expanded Disability Status Scale (EDSS) score.

6. The method of claim 5, wherein the patient had an EDSS score of 5 or less prior to administration of laquinimod.

7. The method of claim 5, wherein the patient had an EDSS score of 5.5 or greater prior to administration of laquinimod.

8. The method of claim 6, wherein confirmed disease progression is at least a 1 point increase of the EDSS score.

9. The method of claim 7, wherein confirmed disease progression is at least a 0.5 point increase of the EDSS score.

10. The method of any one of claims 4-9, wherein the time to confirmed disease progression is increased by 20-60%.

11. The method of any one of claims 4-9, wherein the time to confirmed disease progression is increased by at least 50%.

12. The method of claim 3, wherein the administration of laquinimod is effective to increase the time to confirmed relapse in the human patient.

13. The method of claim 12, wherein the time to confirmed relapse is increased by at least 20%.

14. The method of claim 13, wherein the time to confirmed relapse is increased by at least 30%.

15. The method of claim 3, wherein the administration of laquinimod is effective to reduce brain atrophy in the human patient.

16. The method of claim 15, wherein brain atrophy is reduced by at least 20%.

17. The method of claim 16, wherein brain atrophy is reduced by at least 30%.

18. The method of claim 3, wherein the administration of laquinimod is effective to reduce relapse rate in the human patient.

19. The method claim 18, wherein the relapse rate is reduced by at least 30%.

20. The method claim 19, wherein the relapse rate is reduced by at least 70%.

21. The method of claim 3, wherein the administration of laquinimod is effective to reduce the accumulation of disability in the human patient.

22. The method of claim 21, wherein the accumulation of disability is assessed by the timed 25-foot walk (T25FW).

23. The method of claim 3, wherein the administration of laquinimod is effective to reduce or inhibit progression of the level of fatigue in the human patient.

24. The method of claim 23, wherein the level of fatigue is assessed by the patient's Modified Fatigue Impact Scale (MFIS) score.

25. The method of claim 24, wherein the administration of laquinimod decreased the human patient's MFIS score, compared to a patient not receiving the laquinimod treatment.

26. The method of claim 24 or 25, wherein the administration of laquinimod decreased the human patient's MFIS score, compared to the patient at the start of the laquinimod treatment.

27. The method of any one of claims 24-26, wherein the MFIS score decreased within 24 months of the start of laquinimod treatment.

28. The method of claim 3, wherein the administration of laquinimod is effective to improve or inhibit deterioration of the functional status in the human patient.

29. The method of claim 28, wherein the functional status of the patient is measured by the patient's Short-Form General Health survey (SF-36) Subject-Reported Questionnaire score.

30. The method of claim 29, wherein the administration of laquinimod decreased the human patient's SF-36 score, compared to a patient not receiving the laquinimod treatment.

31. The method of claim 29 or 30, wherein the administration of laquinimod decreased the human patient's SF-36 score, compared to the patient at the start of the laquinimod treatment.

32. The method of any one of claims 29-31, wherein the patient's SF-36 mental component summary score (MSC) is decreased.

33. The method of any one of claims 29-32, wherein the patient's SF-36 physical component summary score (PSC) is decreased.

34. The method of any one of claims 29-33, wherein the SF-36 score is decreased within 24 months of the start of laquinimod treatment.

35. The method of claim 3, wherein the administration of laquinimod is effective to improve or inhibit deterioration of the general health in the human patient.

36. The method of claim 35, wherein the general health of the patient is assessed by the patient's EQ-5D Standardized Questionnaire score.

37. The method of claim 36, wherein the administration of laquinimod increased the human patient's EQ-5D score, compared to a patient not receiving the laquinimod treatment.

38. The method of claim 36 or 37, wherein the administration of laquinimod increased the human patient's EQ-5D score, compared to the patient at the start of the laquinimod treatment.

39. The method of any one of claims 36-38, wherein the EQ-5D score increased within 24 months of the start of laquinimod treatment.

40. The method of claim 3, wherein the administration of laquinimod is effective to reduce MRI-monitored disease activity in the human patient.

41. The method of claim 40, wherein the MRI-monitored disease activity is assessed by the number of GdE-T1 lesions, the number of new T2 lesions, the number of new T1 hypointense lesions (black holes), change in T2 lesions volume, change in GdE-T1 lesions volume or change in T1 hypointense lesions volume (black holes).

42. The method of claim 3, wherein the administration of laquinimod is effective to reduce cognitive impairment in the human patient.

43. The method of claim 42, wherein the cognitive impairment is assessed by the Symbol Digit Modalities Test (SDMT) score.

44. The method of any one of claims 1-43, wherein the patient had disease duration of at least 6 months prior to starting laquinimod treatment.

45. The method of any one of claims 1-44, wherein the laquinimod is administered as monotherapy for multiple sclerosis.

46. The method of any one of claims 1-44, wherein the laquinimod is administered as adjunct therapy with an other multiple sclerosis treatment.

47. The method of claim 46, wherein the other relapsing-remitting multiple sclerosis treatment is administration of interferon beta 1-a, interferon beta 1-b, glatiramer acetate, mitoxantrone, natalizumab, dialkyl fumarate or fingolimod.

48. The method of any one of claims 1-47, wherein the human patient is afflicted with relapsing-remitting multiple sclerosis.

49. A method for treating a human subject by providing neuroprotection to the human subject comprising orally administering to the human subject a daily dose of about 1.2 mg laquinimod or a pharmaceutically acceptable salt thereof so as to thereby treat the human subject by providing neuroprotection to the human subject.

50. The method of claim 49, wherein the administration of laquinimod reduces neuronal dysfunction, reduces neuronal injury, reduces neuronal degeneration, or reduces neuronal apoptosis.

51. The method of claim 50, wherein the administration of laquinimod reduces neuronal dysfunction in the Central Nervous System, reduces neuronal injury in the Central Nervous System, reduces neuronal degeneration in the Central Nervous System, or reduces neuronal apoptosis in the Central Nervous System.

52. A method of treating a human patient afflicted with multiple sclerosis or presenting a clinically isolated syndrome, by increasing the time to confirmed disease progression, increasing the time to confirmed relapse or reducing brain atrophy in the human patient, the method comprising orally administering to the patient laquinimod or a pharmaceutically acceptable salt thereof at a daily dose of about 1.2 mg laquinimod so as to thereby treat the human patient by increasing the time to confirmed disease progression, increasing the time to confirmed relapse or reducing brain atrophy in the human patient.

53. The method of claim 52, wherein the administration of laquinimod is effective to increase the time to confirmed disease progression in the human patient.

54. The method of claim 52, wherein the administration of laquinimod is effective to increase the time to confirmed relapse in the human patient.

55. The method of claim 52, wherein the administration of laquinimod is effective to reduce brain atrophy in the human patient.

56. The method of any one of claims 52-55, wherein the laquinimod is administered as monotherapy for multiple sclerosis.

57. The method of any one of claims 52-55, wherein the laquinimod is administered as adjunct therapy with an other multiple sclerosis treatment.

58. The method of claim 57, wherein the other relapsing-remitting multiple sclerosis treatment is administration of interferon beta 1-a, interferon beta 1-b, glatiramer acetate, mitoxantrone, natalizumab, dialkyl fumarate or fingolimod.

59. The method of any one of claims 52-58, wherein the human patient is afflicted with relapsing-remitting multiple sclerosis.

60. The method of any one of claims 1-59, comprising orally administering to the human patient or subject laquinimod or a pharmaceutically acceptable salt thereof at a daily dose of 1.2 mg laquinimod.

61. The method of any one of claims 1-60, wherein the laquinimod is administered in the form of laquinimod sodium.

62. The method of any one of claims 1-61, wherein the administration is for a period of greater than 24 weeks.

63. The method of any one of claims 1-62, wherein laquinimod is administered in the form of a tablet or a capsule.

64. A pharmaceutical oral unit dosage form of about 1.2 mg laquinimod or a pharmaceutically acceptable salt thereof and a pharmaceutically acceptable carrier for use in treating a human patient afflicted with multiple sclerosis or presenting a clinically isolated syndrome.

65. A pharmaceutical oral unit dosage form of about 1.2 mg laquinimod or a pharmaceutically acceptable salt thereof and a pharmaceutically acceptable carrier for use in treating a human subject by providing neuroprotection to the human subject.

66. A pharmaceutical oral unit dosage form of about 1.2 mg laquinimod or a pharmaceutically acceptable salt thereof and a pharmaceutically acceptable carrier for use treating a human patient afflicted with multiple sclerosis or presenting a clinically isolated syndrome, by increasing the time to confirmed disease progression, increasing the time to confirmed relapse or reducing brain atrophy in the human patient.

67. The pharmaceutical oral unit dosage form of any one of claims 64-66, in the form of a tablet or a capsule.
